(19) United States
(12) Patent Application Publication
(10) Pub. No.: US 2009/0069830 A1
(43) Pub. Date: Mar. 12, 2009

(54) EYE SURGICAL TOOL

(75) Inventors: Maureen L. Mulvihill, Bellefonte, PA (US); David E. Booth, Wyomissing Hills, PA (US); Brian M. Park, State College, PA (US)

Correspondence Address: THE WEBB LAW FIRM, P.C., 700 KOPPERS BUILDING, 436 SEVENTH AVENUE, PITTSBURGH, PA, 15219, US

(73) Assignee: PIEZO RESONANCE INNOVATIONS, INC., Bellefonte, PA (US)

(21) Appl. No.: 12/134,638

(22) Filed: Jun. 6, 2008

Related U.S. Application Data
(60) Provisional application No. 60/933,528, filed on Jun. 7, 2007.

(51) Int. Cl. A61B 17/32

(52) U.S. Cl. 606/171

(57) ABSTRACT

The present invention is directed to a surgical cutting device having a body, a piezoelectric actuator received within and secured to the body and a blade associated with and in communication with the actuator. The actuator is adapted for vibrating at a frequency to produce an oscillating displacement of the blade. A method of operating the surgical cutting device is also provided wherein the cutting device includes an actuator which is adapted for vibrating at a frequency to produce a sinusoidal displacement of the blade in the range of 250-500 μm.

### CROSS-REFERENCE TO RELATED APPLICATION

This application claims the benefit of U.S. Provisional Application No. 60/933,528 filed on Jun. 7, 2007. The subject matter of the prior application is incorporated in its entirety herein by reference thereto.

### BACKGROUND OF THE INVENTION

1. Field of the Invention

The present invention generally pertains to surgical instruments, and more specifically to high-speed electrically driven surgical blades. The invention is applicable to the cutting of skin and other tissues or materials found within the body.

Cataract surgery is the most common surgical procedure in the United States today with close to 2 million procedures performed annually. Ocular keratomes are used to create self-sealing incisions entering through the conjunctiva, scleara or cornea to form clear corneal incisions during cataract surgery. Self-sealing incisions may also be referred to as self-healing incisions as there is no need to cauterize tissue to prevent further tissue damage and bleeding.

In general surgical applications, percutaneous access to tissues and vasculature as well as access through body-surface organ tissues like the conjunctiva and sclera is typically accomplished with non-vibrating cutting and shearing edges. Due in part to the variability of sharpness of conventional metal ophthalmic knife blades, the force required to create an incision into the eye tissue can cause significant tissue trauma, separating stromal layers and causing delamination of the Descemets membrane. As the surgeon applies force through the handle to a non-actuated blade, the point ruptures the surface membrane of the tissue and the edges cut and divide the tissue. Essentially, the blade is resisted by the force of the elastically deforming tissue. The blade is also resisted by the force required to divide the tissue at the cutting edges and the force created by the adhesive bonds between the blade and the tissue.

Several advances have been attempted to reduce the force necessary to penetrate a blade through tissue. Most of these, such as U.S. Pat. No. 6,554,840 (Matsutani et al.) for example, simply reduce the cutting edge to blade thickness ratio to lower the penetration force. Others, such as U.S. Pat. No. 6,547,802 (Nallakrishnan et al.) seek to improve incisions to the eye by maximizing the surface area of the cut with a blade having a wide surface area comprised of two cutting edges disposed at an angle greater than 90°. Meanwhile, U.S. Pat. No. 6,056,764 (Smith) not only changes the blade tip angle, or angle between cutting edges on either side of a sharp tip, but also offers alternative blade materials such as diamond, sapphire, ruby, and cubic zirconia. Additionally, the '764 patent teaches the use of coatings over stainless steel blades to add strength to the blade. Other conventional attempts also disclose applying a surface treatment in the form of a hydrophobic/hydrophilic coating to the blade. However, while some reduction of force may be attained by the aforementioned disclosures, they are limited to only reducing the bulk surface friction between the instrument surface and the tissue surface being cut, and changing the surface area of the blade or changing the coefficient of friction between the surfaces.

One of the problems associated with surface treatment of surgical blades is that the blade sharpness is sacrificed for a lowering of mechanical friction. Also, an associated problem with changing the dimensions of the blade is faster dulling, further resulting in increased friction at the blade-tissue interface. These results only further promote cauterization and do not contribute to reducing the force necessary for penetration.

Another approach to cutting and penetrating through tissue is to sonically or ultrasonically vibrate the cutting edges of a surgical blade. Because piezoelectric ceramics deform when exposed to an electrical input, a phenomenon known as the converse piezoelectric effect, current technologies utilize stacks of piezoelectric material such as lead-zirconate-titanate (PZT) to produce the mechanical, ultrasonic motion. For example, U.S. Pat. No. 4,587,958 (Noguchi) discloses an ultrasonic surgical device that focuses on the application of ultrasonic energy to shatter tissue. Unfortunately, it is apparent from the '958 disclosure that the express purpose of the ultrasonic vibrations applied upon the device is to “exhibit a satisfactory tissue shattering capacity”. As a result, this type of tissue penetration does not minimize scarring, but instead creates a blunt incision by shattering the tissue.

On the other hand, U.S. Pat. No. 5,935,143 (Hood) attempts to minimize the “thermal footprint” of an ultrasonic blade. This is done by using a Langevin or dumbbell type transducer to produce axial motion of the cutting blade, thereby providing tactile feedback and enhanced ergonomics to the surgeon using the blade. The combination of ultrasonic vibration coupled with sinusoidal axial motion of the '143 blade perpendicular to the tissue surface plane also causes coagulation and cauterization of the tissue being incised and, therefore, does not increase the quality of the incision.

While it's been shown in the art that ultrasonically vibrating a blade enhances its sharpness, U.S. Pat. No. 5,324,299 (Davison, et al.) teaches that without proper configuration and design, an ultrasonic blade's “sharpness” is not enhanced when cutting through relatively loose and unsupported tissues. Therefore, the '299 reference teaches ultrasonically driven scalpel blades having a hook tip design which focuses some of the vibration in a particular direction, but does not actually increase the quality of the incision as it serves to enhance coagulation of the tissue being incised. Furthermore, a hooked tip prevents the blade from being optimally tuned for stab type incisions.

Unfortunately, the focus of the improvements of vibrating blades found in the aforementioned prior-alt disclosures were made with little regard to secondary issues related to incising tissue. For example, secondary issues such as those aspects of surgical procedure beyond simply incising the tissue include minimizing the pain experienced by patients during tissue penetration, minimizing scarring and improving wound healing, all of which are the result of having created a high quality incision at a reduced force necessary for cutting, incising, penetrating and the like.

Advancements in the surgical arts have been attempted to address these secondary issues. For instance, it has been shown that oscillating the blade of a surgical tool laterally or parallel to the tissue surface, rather than axially or perpendicular to its surface, may reduce pain during incising. As is disclosed in U.S. Pat. No. 6,210,421 (Bocker, et al.), the lateral motion of the blade against the skin reduces the pressure waves that would otherwise be directed perpendicular to the skin in an axially driven blade, resulting in a smaller number of pain receptors being activated. The '421 patent, however, is directed to a blood lancet which is not optimal for cutting tissue to a depth necessary as in ocular or minimally invasive surgery.

In an attempt to optimize tissue incising, U.S. Pat. No. 6,254,622 (Hood) discloses an ultrasonically driven blade having an unsymmetrical cutting surface which causes an offset center of gravity that creates transverse movement of the blade, perpendicular to the longitudinal axis of the surgical device. The blade, having a low attack angle to form the asymmetric shape that gives the blade a sharp point, is able to then effectively cut both hydrogenous tissue and non-hydrogenous tissue without requiring tension on the cutting medium. The transverse movement of the blade provides an efficient means of transferring the ultrasonic energy directly into the tissue and also moves the blood away from the cutting edge, allowing for a more efficient transfer of ultrasonic energy to the tissue. Unfortunately, the '622 patent relies on a driving frequency from 60,000-120,000 Hz, a frequency range that is generally too high for preserving the soft tissue as it usually causes thermal damage.

In yet another attempt to transform the axial motion of a driving piezoelectric transducer into transverse motion of a surgical blade, U.S. Pat. No. 6,585,745 (Cimino) discloses a split-electrode configuration to drive a bolt-type or Langevin actuator 311. The patent discloses the use of lower frequencies such as 10 kHz in an axial or longitudinal direction, causing a transverse motion of the blade perpendicular to the long axis of the device. While the '745 patent attempts to disclose that the device produces improved cutting, it is inherently flawed as it depends on the split-electrode configuration, which is complex as compared to a single-phase pattern. Because the split-electrode configuration causes the piezoelectric transducers that drive the device to contract on one half and expand on the other, the device is vulnerable to induced stress and cracking, thereby reducing life and efficiency.

Lateral motion of the blade in a surgical tool has also been combined with longitudinal motion, such as that which is described in U.S. Patent Application No. 2005/0234484 A1 (Houser, et al.). While the '484 application discloses that longitudinal ultrasonic vibration of the blade generates motion and heat, thereby assisting in the coagulating of the tissue, the disclosure also recognizes that transverse ultrasonic vibration of the blade offers beneficial results. One such result is a total ultrasonic vibration having an amplitude that is larger and more uniform over a long distance of the blade as compared to surgical blades having only longitudinal vibrations. Yet, the invention relies solely on ultrasonic vibrations, which inherently limits the invention to incising specific tissues only, and not the wide range of tissues that are encountered during a surgical procedure. A weakness of all blades, which are solely ultrasonically driven, is that they atomize the surrounding fluids. Because fluids are broken into small droplets when they encounter a solid mass vibrating at ultrasonic frequencies, the fluids becomes a mobile “mist” of sorts. As droplets, which have a size inversely proportional to the vibrating frequency, the fluid “mist” is similar to that of room humidifiers and also to the droplets created by industrial spray nozzles. One negative aspect of creating a mobile mist during a surgical procedure is that these particles may contain viral or bacterial agents. By ultrasonically vibrating the moisture surrounding unhealthy tissue as it is being incised, it is possible to unknowingly transport the bacterial or viral agent to healthy tissue. It, therefore, is an inherent weakness of ultrasonically driven surgical blades that they increase the chance of spreading disease or infection.

Therefore, a need exists for an improved surgical blade that is able to be vibrated sonically and ultrasonically, reducing the force required to penetrate tissue, and thereby reduces the amount of resulting tissue damage and scarring while also improving wound healing.

### SUMMARY OF THE INVENTION

Transducer technologies that rely on conventional, single or stacked piezoelectric ceramic assemblies for actuation are hindered by the maximum strain limit of the piezoelectric materials themselves. Because the maximum strain limit of conventional piezoelectric ceramics is about 0.1% for polycrystalline piezoelectric materials, such as ceramic lead zirconate titanate (PZT) and 0.5% for single crystal piezoelectric materials, it would require a large stack of cells to approach useful displacement or actuation of, for example, a handheld device usable for processes such as cutting, slicing, penetrating, incising and the like. However, using a large stack of cells to actuate components of a handpiece would also require the tool size to increase beyond usable biometric design for handheld instruments.

Flextensional transducer assembly designs have been developed which provide amplification in piezoelectric material stack strain displacement. The flextensional designs comprise a piezoelectric material transducer driving cell disposed within a frame, platten, end-caps or housing. The geometry of the frame, platten, end caps or housing provides amplification of the transverse, axial, radial or longitudinal motions of the driver cell to obtain a larger displacement of the flextensional assembly in a particular direction. Essentially, the flextensional transducer assembly more efficiently converts strain in one direction into movement (or force) in a second direction.

The present invention comprises a handheld device including a cutting, slicing, incising member which is actuated by a flextensional transducer assembly. For example, the flextensional transducer assembly may utilize flextensional cymbal transducer/actuator technology or amplified piezoelectric actuator (APA) transducer technology. The flextensional transducer assembly provides for improved amplification and improved performance which are above that of conventional handheld devices. For example, the amplification may be improved by up to about 50-fold. Additionally, the flextensional transducer assembly enables handpiece configurations to have a more simplified design and a smaller format.

The present invention relates generally to a minimally invasive surgical blade for the cutting and incising of various materials and tissues within a body. Specifically, the present invention is a handpiece comprising a body, at least one piezoelectric transducer driver disposed within the body, a motion transfer adaptor and a surgical blade for cutting, incising and penetrating.

The invention is also a method for cutting, incising and penetrating tissues or other materials found within a patient's body using a handheld surgical tool comprising a body, at least one piezoelectric transducer disposed within the body, a motion transfer adaptor having at least a distal end and a proximal end, and a surgical blade.

The method includes driving the at least one piezoelectric transducer disposed within a body of the handheld surgical tool sinusoidally in a frequency range of 10-1000 Hertz (Hz) and at an electric field in the range of about 300-500 V/mm. Specifically, the blade is driven sinusoidally at such a frequency and displacement so as to attain a peak velocity in the range of 0.9-2.5 m/s, more preferably in the range of 1.0-2.5 m/s and most preferably in the range of 1.5-2.0 m/s. The sinusoidal vibrations are transferred mechanically to the motion transfer adapter coupled at the proximal end to the at least one piezoelectric transducer. The vibrations are further transferred mechanically to the surgical blade attached to a proximal end of the motion transfer adaptor. The surgical blade is configured in such a manner so as to oscillate in a direction that comprises an in-plane motion. In particular, the in-plane motion comprises motion that is primarily in one plane. Most preferably, the surgical blade of the present invention is parallel to the surface of the tissue which is being incised, cut, penetrated or the like, by the blade. The in-plane motion is such a motion that is primarily perpendicular to the long axis of the device handle. In other words, the sinusoidal vibrations are an axial driving motion produced parallel to a hypothetical, centrally located axis which extends through a distal end and through a proximal end of a surgical tool's handle portion. The axial driving motion is transposed into lateral motion, perpendicular to the direction of the originating sinusoidal vibrations. It is an object of this invention to reduce tissue deformation, thereby giving superior shaped flap peripheries and flap or stromal bed apposition in ophthalmologic surgical procedures.

In one embodiment, the piezoelectric transducer is a standard bimorph actuator or a variable thickness bimorph similar to but not limited to, those configurations which are described by Cappalleri, D. et al in “Design of a PZT Bimorph Actuator Using a Metamodel-Based Approach”, Transactions of the ASME, Vol. 124 June 2002 and is hereby incorporated by reference.

In another embodiment, the piezoelectric transducer is a cymbal transducer/actuator similar to, but not limited to, that which is described in U.S. Pat. No. 5,729,077 (Newnham) and is hereby incorporated by reference.

In one embodiment, the piezoelectric transducer is a Langevin or dumbbell type transducer similar to, but not limited to, that which is disclosed in U.S. Patent Publication No. 2007/0063618 A1 (Bromfield), which is hereby incorporated by reference.

In yet another embodiment, the piezoelectric transducer is an APA transducer similar to, but not limited to, that which is described in U.S. Pat. No. 6,465,936 (Knowles et al.) and is hereby incorporated by reference.

These and other features of this invention are described in, or are apparent from, the following detailed description of various exemplary embodiments of this invention.

### BRIEF DESCRIPTION OF THE DRAWINGS

Exemplary embodiments of this invention will be described with reference to the accompanying figures.

FIG. 1 is a graph illustrating the reduction of force response.

FIG. 2 is a perspective view of a first embodiment of the handheld surgical device.

FIG. 3A is a cross sectional view of the piezoelectric bender-type actuator shown in FIG. 2.

FIG. 3B is a perspective view of the piezoelectric bender-type actuator shown in FIG. 3A.

FIG. 4 is a cross section view of a variable thickness unimorph type actuator.

FIG. 5 is a visual representation of an example surgical blade of the present invention undergoing sinusoidal, lateral motion.

FIG. 6 is a cross-sectional view of a second embodiment of the handheld surgical device.

FIG. 7 is a cross-sectional view of a third embodiment of the handheld surgical device.

FIG. 8 is a cross-sectional view of a fourth embodiment of the handheld surgical device.

### REFERENCE LABELS

A Static blade force curve
B Vibrating blade force curve
D1 Displacement distance
D2 Displacement distance
W Blade width
TCW Total Cut Width
BA Hypothetical Bender long axis
HLA Hypothetical Long Axis
100 Bender actuated surgical tool
110 Body
111 Bimorph piezoelectric transducer/actuator
111′ Variable Thickness unimorph piezoelectric actuator
112 Piezoelectric plate
113 Bender support bar
113′ First side surface
113″ second side surface
114 Bender motion constraint
115 Bolt through hole
115′ Bolt
116 Support Surface
117 Bender distal end
118 Bender proximal end
119 Blade
119′ first blade displacement position
119″ second blade displacement position
120 Blade collar
121 Collar Attachment node
122 first cutting edge
122′ first cutting edge displacement position
123 second cutting edge
123′ second cutting edge displacement position
124 blade tip
125 first blade ear
125′ first blade ear positive displacement position
125″ first blade ear negative displacement position
126 second blade ear
126′ second blade ear positive displacement position
126″ second blade ear negative displacement position
127 first piezoplate stack
127a first layer
127a′ first layer upper surface
127a″ first layer bottom surface
127b second layer
127b′ second layer upper surface
127b″ second layer bottom surface
127c third layer
127c′ third layer upper surface
127c″ third layer bottom surface
127d fourth layer
127d′ fourth layer upper surface
127d″ fourth layer bottom surface
128 second piezoplate stack
129 first conducting electrical plate
129′ second conducting electrical plate
129″ third conducting electrical plate
131 ground connector
132 positive connector
133 negative connector
134 body proximal end
135 body distal end
200 cymbal actuated surgical tool
210 body
211 cymbal actuator/actuator
212 piezoelectric ceramic disc
213 first end-cap
214 second end-cap
215 dual beveled angled slit blade
216 blade neck
217 attachment node
218 motion constraining neck yoke
219 set screw
220 hypothetical long axis
300 Langevin actuated surgical tool
310 body
311 Langevin actuator
312 Langevin support collar
313 Piezoelectric ceramic discs
314 backing portion
315 Horn portion
316 compression bolt
317 Attachment node
318 Motion transfer adaptor
319 blade
320 Hypothetical long axis
400 APA transducer driven surgical tool
410 Body
411 APA transducer
412 Piezoelectric cell
413 Frame
414 Frame top wall
415 frame bottom wall
416 spacing member
417 blade neck
418 Motion constraining yoke
419 Blade
420 Blade Neck

### DETAILED DESCRIPTION OF THE INVENTION

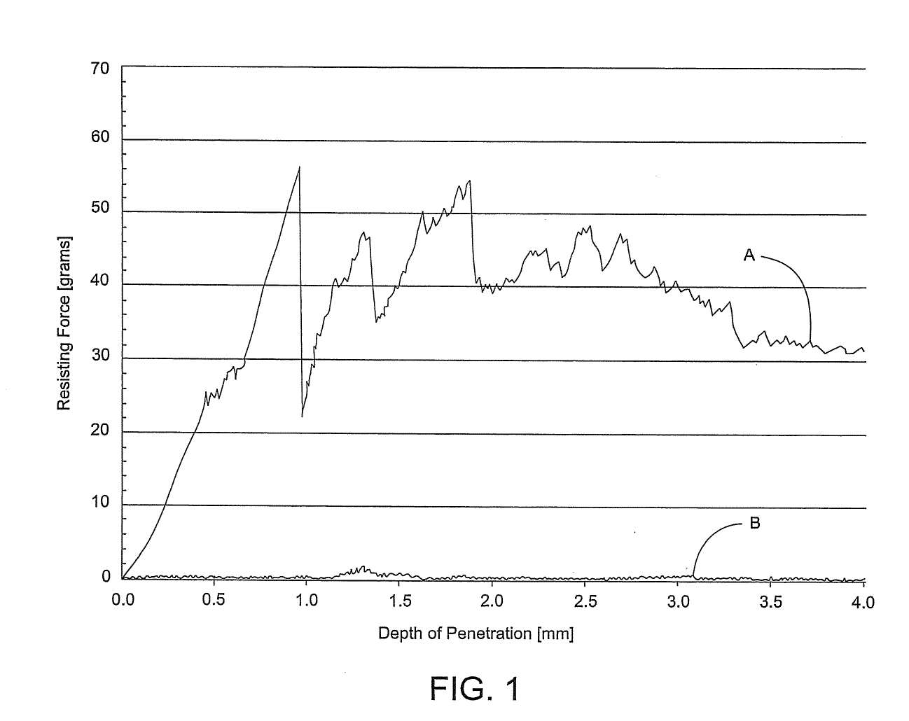

The preferred embodiments of the present invention are illustrated in FIGS. 1 through 8 with the numerals referring to like and corresponding parts.

The effectiveness of the invention as described, for example, in the aforementioned preferred embodiments, relies on the reduction of force principle in order to optimize incising, cutting or penetrating through tissue or materials found within the body. Essentially, when tissue is incised, cut, penetrated or separated by the high-speed operation of the surgical blade of the present invention, the tissue is held in place purely by its own inertia. In other words, a reduction of force effect is observed when a knife blade, for example a slit knife blade, is vibrated with an in-plane motion during the incision process and enough mechanical energy is present to break adhesive bonds between tissue and blade. The threshold limits of energy can be reached in the sonic or ultrasonic frequency ranges if the necessary amount of blade displacement is present.

To exploit the reduction of force effect, the surgical blade of the present invention is designed such that the blade attains a short travel distance or displacement, and vibrates sinusoidally with a high cutting frequency. Utilizing the various device configurations as described in the aforementioned embodiments, it has been determined that the sinusoidal motion of the blade must include at least a peak velocity in the range of 0.9-2.5 m/s, more preferably between 1.0-2.25 m/s and most preferably at a velocity of 1.5-2.0 m/s. For example, FIG. 1 shows a graphical representation of the resisting force versus depth of a surgical blade penetrating into material. In FIG. 1, the curve labeled A represents data for a blade in an “off” or non-vibrating condition, and the curve labeled B represents data for a surgical tool having a blade that is vibrated at 450 Hz at and a displacement of 500 μm. As is apparent from FIG. 1, curve A shows that without being vibrated, the force necessary to penetrate into a material is much higher than that for a blade being vibrated, such as that represented by curve B.

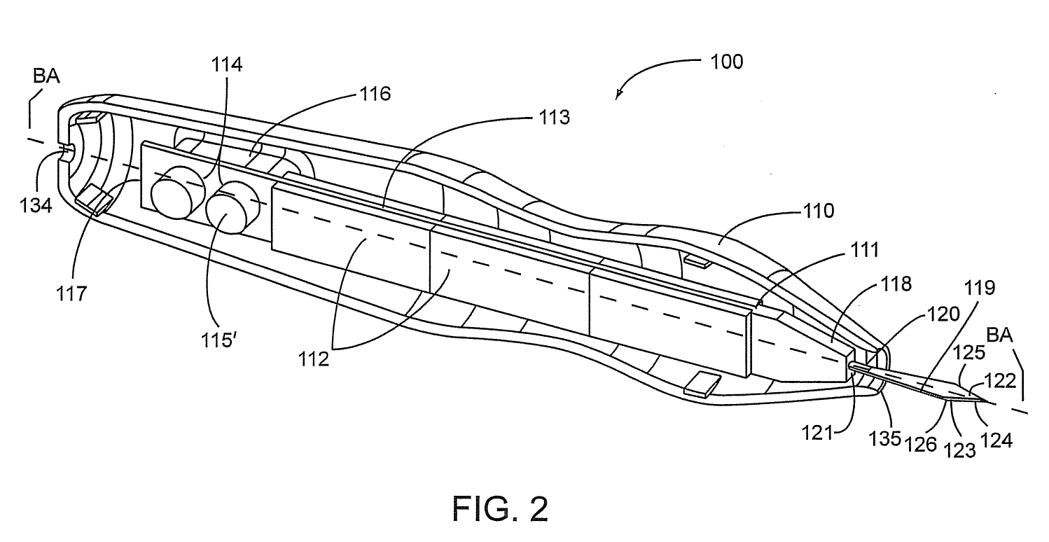

In a first embodiment of the present invention as shown in FIG. 2, a bender actuated surgical tool 100 comprises a body 110, and a bimorph piezoelectric transducer/transducer/actuator 111 disposed within body 110. The bimorph piezoelectric transducer/transducer/actuator 111 comprises at least one piezoelectric ceramic plate 112, but preferably comprises more than one of piezoelectric ceramic plates 112 attached longitudinally upon at least one side of a bender support bar 113. The bender support bar 113 comprises a distal end 117 and a proximal end 118, with a bender motion constraint 114 at the distal end 117. The bender motion constraint 114 attaches bender support bar 113 to surface 116 of the body 110. In one embodiment, the bender motion constraint 114 of the present embodiment comprises at least one thru-hole 115 (not visible in this figure) and a bolt 115′ passing at least partly through the bender support bar 113 and into an attachment slot (not shown) formed on support surface 116. The attachment slot may be, for example, a threaded hole or the like. The bender actuated surgical tool 100 further comprises a blade 119 having a collar 120. The blade collar 120 is directly and mechanically attached to the proximal end 118 of bender support bar 113 at collar attachment node 121. Blade 119 may preferably comprise first cutting edge 122, second cutting edge 123, blade tip 124, first blade ear 125 and second blade ear 126. Collar attachment node 121 may comprise a threaded slot, compression slot, ¼″—cam lock slot, or the like. The bender actuated surgical tool 100 of the present invention also comprises a hypothetical long axis BA which is oriented centrally to rim through a distal end 135 a proximal end 134 of body 110, further passing through the centers of each of body 110, piezoelectric transducer/actuator 111 and blade 119. Blade tip 124 is located externally to body 110.

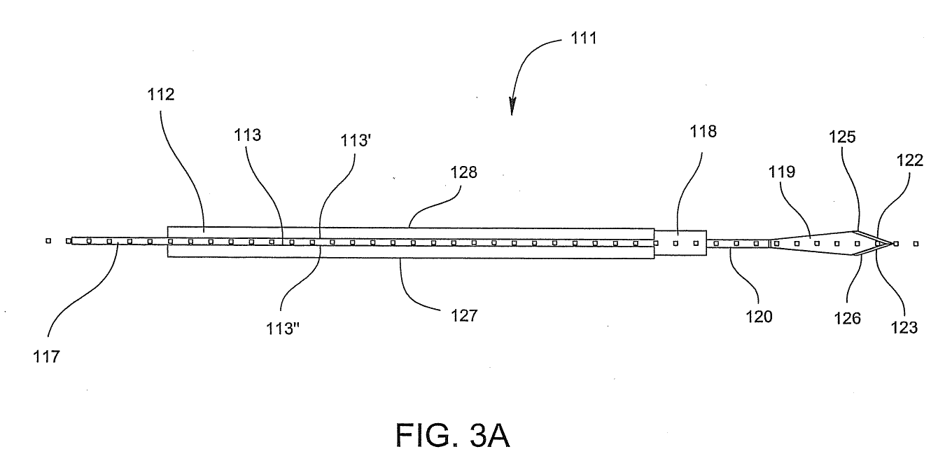

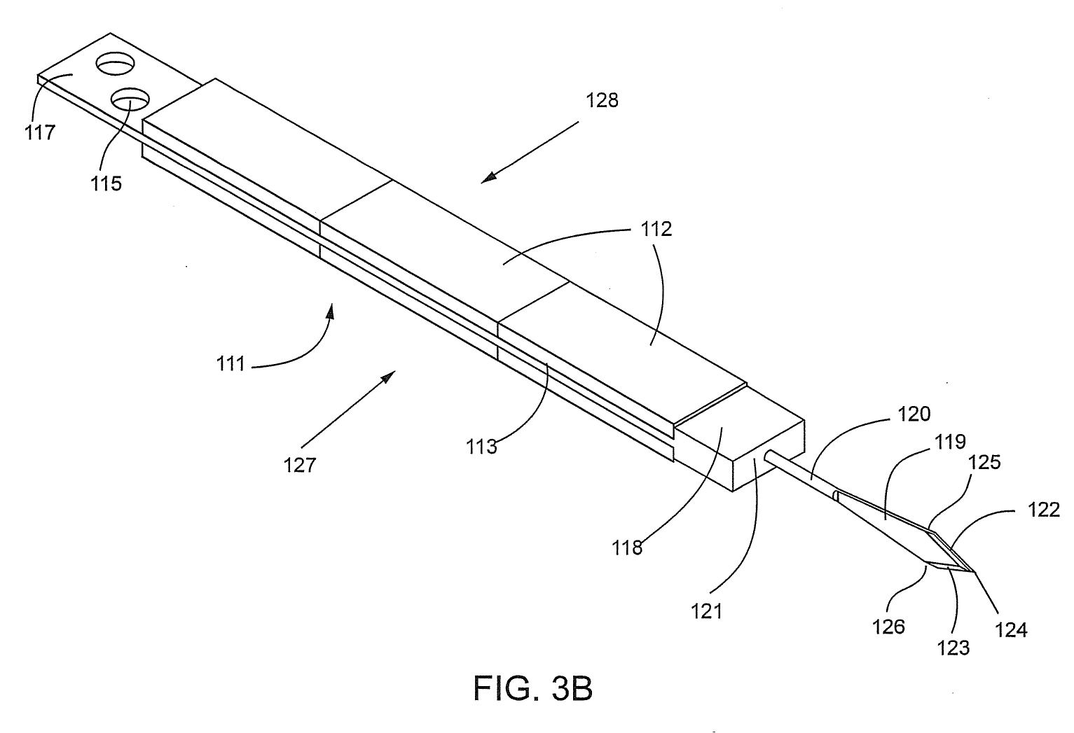

Now, with respect to FIG. 3a, a cross-section of the bimorph piezoelectric transducer/actuator 111 of the bender actuated surgical tool 100 of FIG. 2 is described. Preferably, the bimorph transducer/actuator 111 comprises at least one layer of a plurality of piezoelectric plate 112 formed side by side, each plate being formed longitudinally on, against, and in direct physical and electrical contact to a first side surface 113′ of bender support bar 113, thereby forming first piezoplate stack 127. The bimorph piezoelectric transducer/actuator 111 may also comprise a second piezoplate stack 128 configured in a similar fashion as the first piezoplate stack 127 except each of ceramic plate 112 being formed on, against and in direct physical and electrical contact to a second side surface 113″ formed opposite to the first side surface 113′ of bender support bar 113.

With respect to FIG. 3b, a perspective view of an embodiment of the bimorph piezoelectric transducer/actuator 111 with the blade 119 of the bender actuated surgical tool 100 of FIG. 2 is described. At least one, but preferably two or more of thru-hole 115 are located at distal end 117 of bender support bar 113. A plurality of piezoelectric plates 112 formed side by side, each plate being formed longitudinally on, against and in direct physical and electrical contact to a first side surface 113′ of bender support bar 113, thereby forming first piezoplate stack 127. Again, the bimorph piezoelectric transducer/actuator 111 may also comprise a second piezoplate stack 128 configured in a similar fashion as the first piezoplate stack 127 except piezoelectric plate 112 being formed on, against and in direct physical and electrical contact to a second side surface 113″ formed opposite to the first side surface 113′ of bender support bar 113.

Returning to FIG. 2, electrical contact is made to each of piezoelectric plates 112 of either first piezoplate stack 127 or second piezoplate stack 128, but more preferably both first piezoplate stack 127 and second piezoplate stack 128, by contact leads (not shown) connected to an external circuit (also not shown) so as to actuate the bimorph piezoelectric transducer/actuator 111, with a separate electrical lead attached to the bender bar 113 as a ground electrode. Upon electrical activation of either first piezoplate stack 127 or second piezoplate stack 128, but more preferably upon activation of both first piezoplate stack 127 and second piezoplate stack 128, by an externally applied alternating current, bender bar 113 experiences a compressive force at its first side surface and a tensional force on its second side surface as a result of compression and expansion of the first piezoplate stack 127 and second piezoplate stack 128, respectively, during one cycle of the applied current. Bender bar 113 then experiences a tensional force at its first side surface and a compressive force on its second side surface as a result of expansion and compression of the first piezoplate stack 127 and second piezoplate stack 128, respectively, during the opposite cycle of the applied current. Thereby because proximal end 118 of bimorph transducer/actuator 111 is fixedly attached to body 110 at support surface 116 by bender motion constraint 114, therefore, most importantly, first blade ear 125 and second blade ear 126 are oriented opposite to one another on blade 119 so as to be formed on either side of the aforementioned hypothetical axis, corresponding to the first side surface 113′ and the second side surface 113″ of bender bar 113, respectively. In this way, when the bimorph piezoelectric actuator oscillates upon application of an AC current to electrically activate the first piezoplate stack and second piezoplate stack, a hypothetical first tangential vector passing through first blade ear 125 and hypothetical second tangential vector passing through second blade ear 126 are both parallel at any given point in time to a third hypothetical tangential vector corresponding to a radius of curvature defined by the motion at the blade tip 124 with respect to a fixed position of proximal end 118 held in place by bender motion constraint 114.

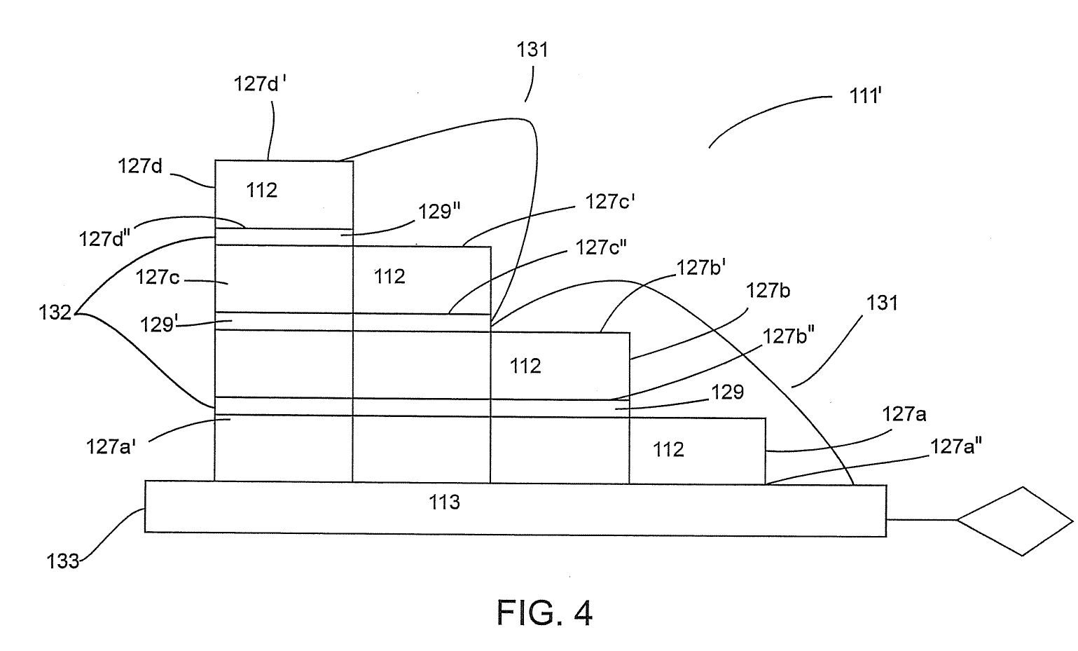

While the actuator of the bender actuated surgical tool has been described with respect to a bimorph type actuator, a unimorph type actuator may easily replace the bimorph piezoelectric transducer 111. In essence, when the bimorph piezoelectric transducer 111 comprises at least one layer of at least one of piezoelectric plate 112 formed side by side, each plate being formed longitudinally against and in direct physical contact to a first side surface 113′ of bender support bar 113 so as to form first piezoplate stack 127, and second piezoplate stack 128 is not formed, the piezoelectric transducer is a unimorph piezoelectric transducer. Furthermore, as shown in FIG. 4, a unimorph piezoelectric transducer may be a variable thickness unimorph piezoelectric transducer 111′. Variable thickness unimorph piezoelectric transducer 111′ comprises a plurality of stacked layers, each formed of at least one of piezoelectric plate 112. In the case that a layer comprises a plurality of piezoelectric plate 112, each plate is formed side by side, and longitudinally along the length of a bender support bar 113. The plurality of layers are further formed such that each additional layer is shorter in length than the previously stacked layer, usually by at least the length of one piezoelectric plate 112, with a conductive plate being formed between each layer. For example, as shown in FIG. 4, first layer 127a having an upper surface 127a′, and a bottom surface 127a″ opposite upper surface 127a′, comprises four piezoelectric plates 112 formed side by side and longitudinally with respect to the length of bender support bar 113, and with bottom surface 127a″ being in direct physical and electrical contact to first side surface 113′ of bender support bar 113. A first conducting electric plate 129 is formed in direct physical and electrical contact to upper surface 127a′. A second layer 127b having an upper surface 127b′ and a lower surface 127b″ opposite upper surface 127b′, comprises three piezoelectric ceramic plates 112 formed side by side and longitudinally with respect to the length of bender support bar 113, and with lower surface 127b″ being in direct physical and electrical contact to first electrical plate 129 at a surface opposite to the interface formed by 127a′/129. A second conducting electrical plate 129′ is formed in direct physical and electrical contact to upper surface 127b′. A third layer 127c having an upper surface 127c′ and a lower surface 127c″ opposite to upper surface 127c′, comprises two piezoelectric ceramic plates 112 formed side by side and longitudinally with respect to the length of bender support bar 113, and with lower surface 127c″ being in direct physical and electrical contact to second electrical plate 129′at a surface opposite to 127b′/129′. A third conducting electrical plate 129″ is formed in direct physical and electrical contact to upper surface 127c′. A fourth layer 127d having an upper surface 127d′ and a lower surface 127d″ opposite to upper surface 127c′, comprises one of piezoelectric plate 112 formed with lower surface 127d″ in direct physical and electrical contact third conducting electrical plate 129″ at a surface opposite to 127c′/129″. Additional features of the functional variable thickness unimorph transducer 111′ include electrical leads necessary for connecting the transducer to an external circuit. The electrical leads comprise a ground connector 131 electrically connecting the upper surface 127d′ of fourth layer 127d to second electrical plate 129′ and also to the bender support bar 113. The electrical leads further comprise positive connector 132 which electrically connects an external circuit (not shown) to third electrical plate 129″ and first electrical plate 129. A negative connector 133 electrically connects the external circuit to bender support bar 113.

The bimorph piezoelectric transducer 111 may also be of a variable thickness type, so long as in the case of either the first piezoplate stack 127 or second piezoplate stack 128 comprise more than one layer of piezoelectric ceramic plate 112, with each additional layer being shorter in length than the previously stacked layer and a conductive plate being formed between each layer. In other words, a variable thickness bimorph piezoelectric transducer may be formed in a similar fashion as prescribed to unimorph piezoelectric transducer 111′ with the exception that the multiplicity of layers of piezoelectric ceramic plates is symmetrically formed on second side surface 113″ of bender support bar 113.

The functional performance of the surgical tool is driven by the piezoelectric elements section. Piezoelectric ceramic elements, such as each of one or more piezoelectric ceramic plate 112 are capable of precise, controlled displacement and can generate energy at a specific frequency. The piezoelectric ceramics expand when exposed to an electrical input, due to the asymmetry of the crystal structure, in a process known as the converse piezoelectric effect. Contraction is also possible with negative voltage. Piezoelectric strain is quantified through the piezoelectric coefficients d33, d31, and d15, multiplied by the electric field, E, to determine the strain, x, induced in the material. Ferroelectric polycrystalline ceramics, such as barium titanate (BT) and lead zirconate titanate (PZT), exhibit piezoelectricity when electrically poled. Simple devices composed of a disk or a multilayer type directly use the strain induced in a ceramic by the applied electric field. Acoustic and ultrasonic vibrations can be generated by an alternating field tuned at the mechanical resonance frequency of a piezoelectric device. Piezoelectric components can be fabricated in a wide range of shapes and sizes. A piezoelectric component may be 2-5 mm in diameter and 3-5 mm long, possibly composed of several stacked disks or plates. The exact dimensions of the piezoelectric component are performance dependent.

The piezoelectric ceramic material may be comprised of at least one of lead zirconate titanate (PZT), multilayer PZT, polyvinylidene difluoride (PVDF), multilayer PVDF, lead magnesium niobate-lead titanate (PMNPT), multilayer PMN, electrostrictive PMN-PT, ferroelectric polymers, single crystal PMN-PT (lead zinc-titanate), and single crystal PZN-PT.

Bender bar 113 may comprise a metal such as stainless steel, titanium, or another conductive material also having high rigidity.

Returning to FIG. 2, upon application of an external AC current at a predetermined frequency to the first or second, or both the first and second piezoplate stacks, bimorph piezoelectric transducer/actuator 111 reactively changes shape in a sinusoidal fashion such that the relative position of blade 119 with respect to say, a fixed position of a point on distal end 117 held in place by bender motion constraint 114 changes by a predetermined displacement. Because the AC current is a sinusoidal signal, the result of activating the piezoelectric ceramic plates is a sinusoidal, back and forth motion of the piezoelectric actuator, and the blade 119, with the blade achieving a peak velocity at a central location of the sinusoidal motion.

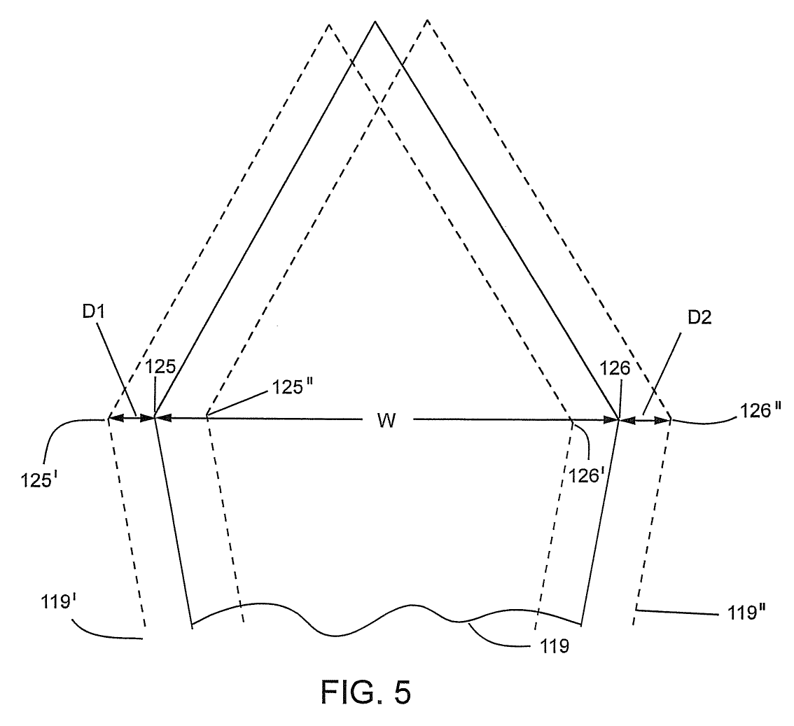

As depicted in FIG. 5, blade 119 appears at a location defined by the dark solid line at a moment directly preceding the application of an external AC current to the surgical blade of the invention. Blade 119 also appears at the location defined by the dark solid line upon attaining a peak velocity once motion has reached steady state after application of an external AC current to the surgical blade of the present invention. Correspondingly, during the positive cycle of an externally applied sinusoidal AC current signal, blade 119 appears at a location defined by the dotted-dashed line as first blade displacement position 119′ while appearing at a location defined by the dashed line as second blade displacement position 119″ during the negative cycle. In other words, blade 119 is displaced by a distance D1, during a positive cycle of the applied AC current at a predetermined frequency to a location defined by blade displacement position 119′. Alternatively, blade 119 is displaced by distance D2 during a negative cycle of the externally applied AC current at a predetermined frequency to a location defined by blade displacement position 119′. Moreover, during for example the positive cycle of an externally applied sinusoidal AC current signal at a predetermined frequency, first blade ear 125 and second blade ear 126 are displaced by distance D1 to locations defined by first blade ear positive displacement position 125′ and second blade ear positive displacement position 126′, respectively. Correspondingly, during the negative cycle of the applied AC current signal, first blade ear 125 and second blade ear 126 are displaced by displacement distance D2 to locations defined by first blade ear negative position 125″ and second blade ear negative displacement position 126″. Ideally, displacement D1 and displacement D2 are approximately equivalent and equal to a distance in the range of 500-750 micrometers. Because the distance between first blade ear 125 and second blade ear 126 across the width of blade 119 is length W, the total distance traveled during a complete cycle of the externally applied AC current signal is W+D1+D2 corresponding to a total cut width TCW.

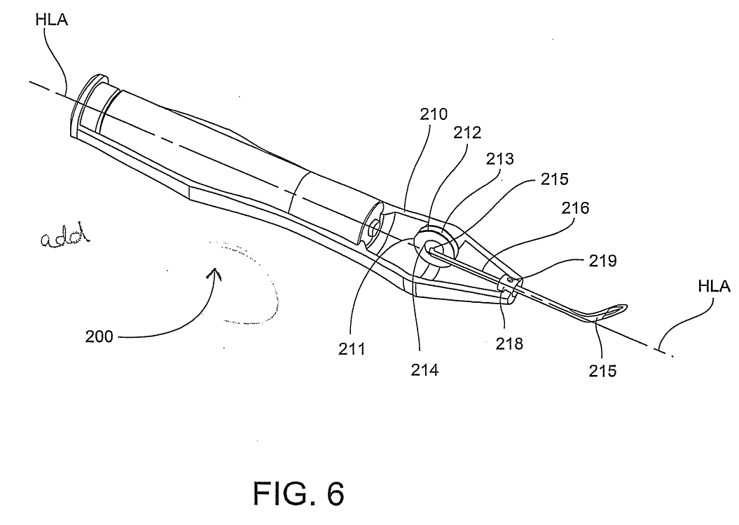

In a second embodiment, the surgical tool of the present invention can be a cymbal actuated surgical tool 200 as shown in FIG. 6. Surgical tool 200 comprises a body 210 and a cymbal actuator 211 which further comprises a piezoelectric ceramic disc 212 stacked between a first end-cap 213 and a second end-cap 214. The first end-cap 213 is fixedly attached to the body 210. Additionally, surgical tool 200 comprises a blade such as a dual beveled angled slit split blade 215. A blade neck 216 is coupled at one end to the second end-cap 214 at attachment node 217, and the blade at an opposite end. A motion constraining yoke 218 is attached to the blade neck at a location between the blade and the attachment node. In one configuration, the motion constraining yoke 218 has a cylindrical shape having an outer diameter with a hollow center defining an inner diameter. The blade neck may be connected to the motion constraining yoke at the inner diameter while the outer diameter is attached to a proximal end of the body 210 such that it is fixedly held in place. For example, the blade neck 216 may be connected to the inner diameter of the motion constraining yoke and held in place by a threaded set screw 219 which passes through the yoke, from the outer diameter to the inner diameter. The set screw compresses at least a portion of the blade neck against at least a portion of the inner diameter surface of the yoke. A hypothetical long axis HLA runs longitudinally in a direction corresponding to the length of the device.

As shown in FIG. 6 the cymbal actuator 211 is a type of flextensional transducer assembly including a piezoelectric ceramic disc 212 disposed within end-caps 213 and 214. The end-caps 213 and 214 enhance the mechanical response to an electrical input, or conversely, the electrical output generated by a mechanical load. Details of the flextensional cymbal transducer/actuator technology is described by Meyer Jr., R. J., et al., “Displacement amplification of electroactive materials using the cymbal flextensional transducer”, Sensors and Actuators A 87 (2001), 157-162. By way of example, a Class V flextensional cymbal transducer/actuator has a thickness of less than about 2 mm, weighs less than about 3 grams and resonates between about 1 and 100 kHz depending on geometry. With the low profile of the cymbal design, high frequency radial motions of the piezoelectric material are transformed into low frequency (about 20-50 kHz) displacement motions through the cap-covered cavity. An example of a cymbal transducer/actuator is described in U.S. Pat. No. 5,729,077 (Newnham et al.) and is hereby incorporated by reference. While the end-caps shown in the figures are round, they are not intended to be limited to only one shape or design. For example, a rectangular cymbal end-cap design is disclosed in Smith N. B., et al., “Rectangular cymbal arrays for improved ultrasonic transdermal insulin delivery”, J. Acoust. Soc. Am. Vol. 122, issue 4, October 2007. Cymbal transducer/actuators take advantage of the combined expansion in the piezoelectric charge coefficient d₃₃ (induced strain in direction 3 per unit field applied in direction 3) and contraction in the d₃₁ (induced strain in direction 1 per unit field applied in direction 3) of a piezoelectric material, along with the flextensional displacement of the end-caps 213 and 214, which is illustrated in FIG. 6. The design of the end-caps 213 and 214 allows both the longitudinal and transverse responses to contribute to the strain in the desired direction, creating an effective piezoelectric charge constant (d_{eff}) according to the formula, d_{eff}=d₃₃+(−A*d₃₁). Since d₃₁ is negative, and the amplification factor (A) can be as high as 100 as the end-caps 213 and 214 bend, the increase in displacement generated by the cymbal compared to the piezoelectric material alone is significant. The end-caps 213 and 214 can be made of a variety of materials, such as brass, steel, or KOVAR®, a nickel-cobalt ferrous alloy compatible with the thermal expansion of borosilicate glass which allows direct mechanical connections over a range of temperatures, optimized for performance and application conditions, a registered trademark of Carpenter Technology Corporation. The end-caps 213 and 214 also provide additional mechanical stability, ensuring long lifetimes for the cymbal transducer/actuators.

The cymbal transducer/actuator 211 drives the dual beveled angled slit split blade 215. When activated by an AC current, the cymbal transducer/actuator 211 vibrates sinusoidally with respect to the current's frequency. Because end-cap 213 is fixed to an inner sidewall of body 210, when transducer 211 is activated, end-cap 214 moves with respect to the body in a direction perpendicular to the hypothetical long axis HLA of the surgical tool. This motion of end-cap 214 is transferred at the attachment node 217 through blade neck 216 and finally to slit split blade 215 which is displaced in a lateral direction to longitudinal axis HLA. Further, the displacement of slit split blade 215 is amplified relative to the displacement originating at piezoelectric ceramic disc 212 when it compresses and expands during activation due in part to the amplification caused by the design of end-caps 213 and 214. An amplification of the motion originating at the piezoelectric ceramic disc 212 and terminating with a displacement of split blade 215 can further be attributed to the combination of yoke 218 and blade neck 216 acting as a fulcrum and arm of a lever. For example, the piezoelectric ceramic disc 212 alone may only displace by about 1-2 microns, but attached to the end-caps 213 and 214, the cymbal transducer/actuator 211 as a whole may generate up to about 1 kN (225 lb-f) of force and about 80 to 100 microns of displacement. This motion is further transferred through the blade neck 216 and yoke 218 as an amplified lateral displacement of split blade 215 of 100-300 microns. For cases requiring higher displacement, a plurality of cymbal transducer/actuators 211 can be stacked end-cap-to-end-cap to increase the total lateral displacement of the split blade 215.

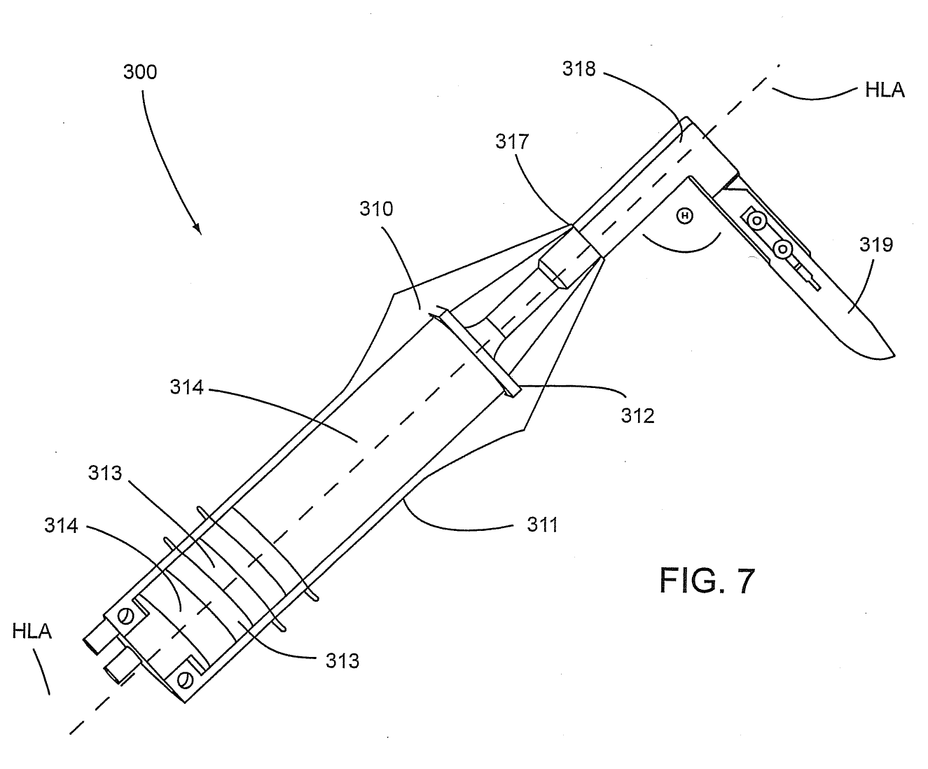

Turning the attention over to FIG. 7, a third embodiment of the invention is shown as a Langevin actuated surgical tool 300. Langevin style transducers have a stack of piezoelectric ceramic discs 313 as shown in FIG. 7. In this embodiment, the surgical tool 300 comprises a body 310 and a conventional Langevin actuator 311 disposed within the body and fixedly held in place at body support collar 312. The Langevin actuator comprises at least one, but preferably more than one piezoelectric ceramic disc 313, a backing portion 314, a horn portion 315 and a compression bolt 316. Horn portion 315 terminates at a proximal end of body 310, and comprises an attachment node 317, which allows a motion transfer adaptor 318 to be mechanically connected to the Langevin actuator. The motion transfer adaptor 318 at one end is functionally attached to attachment node 317 while a blade 319 is attached at another end. A hypothetical long axis HLA runs continuously through the center of each of a distal portion of body 310, a center portion of backing portion 314, compression bolt 316, horn 315, the proximal end of body 310 and at least the center of part of motion transfer adaptor 318. Additionally, motion transfer adaptor comprises a bend having an angle of between 20-90°, which allows the vibrations caused by the activation of ceramic discs 313 to be transferred into a displacement of the blade 319 that is useful for cutting.

In other words, again referring to FIG. 7, when an alternating electric current is applied through the piezoelectric ceramic discs 313, the result is an alternating motion in a direction defined by the displacement of the ceramic discs 313 transferred through the horn 315 and terminating at the tip of the blade 319. The alternating motion results in a reciprocating displacement of the blade 319 relative to the Langevin actuator 311 which is held in place by the body 310 at body support 312. Essentially, with the Langevin actuator 311 fixed to the body 310, the horn 315 communicates this motion to motion transfer adaptor member 318 which in turn communicates motion to the blade 319.

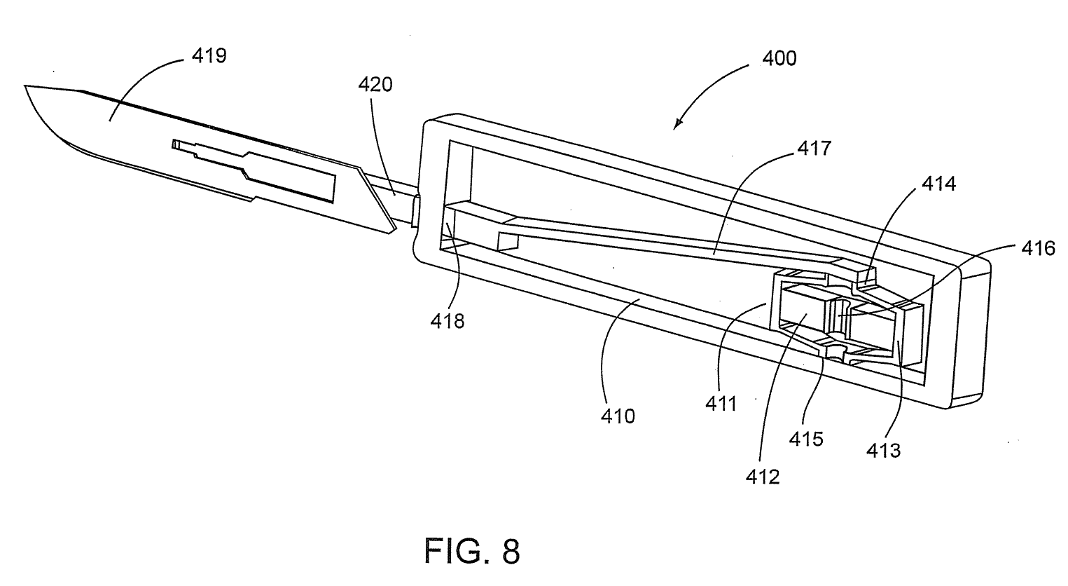

In a fourth embodiment of the present invention, an APA transducer driven surgical tool 400 is shown in FIG. 8. The APA transducer driven surgical tool 400 comprises a body 410, an APA transducer 411, a blade neck 417 attached to the APA transducer, a motion constraining yoke 418, a blade 419 and a blade neck 420. As shown in FIG. 8, the APA transducer 411 is a flextensional transducer assembly including a cell 412 housed within a flexible frame 413. The transducer cell 412 may include a spacing member separating at least two stacks of piezoelectric material. The flextensional transducer cell expands and contracts in one direction to cause movement in the frame. The frame 413 may additionally include either an elbow at the intersection of walls or corrugated pattern along the top and bottom walls, 414 and 415 respectively, of the assembly frame.

In operation, the cell 412 expands during the positive cycle of an AC voltage, which causes top wall 414 and bottom wall 415 of the frame 413 to move inward. Conversely, the transducer cell 412 moves inward during the negative AC cycle, resulting in an outward displacement of the top 414 and bottom 415 walls of the frame 413. However, in the present embodiment, bottom wall 414 is fixedly attached to body 410 so that any movement in the cell will result in only a relative motion of top wall 415 with respect to the body 410 and bottom wall 414. Furthermore, a blade neck 417 is coupled to the top wall 415 on one end, and coupled to a blade 419 at an opposite end. A motion constraining yoke 418 attached to the walls of an opening at a distal end of body 410 serves to constrain blade neck 417 in a similar fashion as the yoke described in FIG. 6.

Two examples of applicable APA transducers are the non-hinged type, and the grooved or hinged type. Details of the mechanics, operation and design of an example hinged or grooved APA transducer are described in U.S. Pat. No. 6,465,936 (Knowles et al.), which is hereby incorporated by reference in its entirety. An example of a non-hinged APA transducer is the Cedrat APA50XS, sold by Cedrat Technologies, and described in the Cedrat Piezo Products Catalogue “Piezo Actuators & Electronics” (Copyright ®Cedrat Technologies June 2005).

While the above described embodiments of the present invention are made with respect to a handheld surgical device having a vibrating blade and utilizing a bender-type, cymbal type, Langevin type or APA type transducer assembly for actuation, the present invention is not limited to these transducer assemblies. Generally, any type of motor comprising a transducer assembly, further comprising a mass coupled to a piezoelectric material, the transducer assembly having a geometry which upon actuation amplifies the motion in a direction beyond the maximum strain of the piezoelectric material, would also fall within the spirit and scope of the invention.

From the above description, it may be appreciated that the present invention provides significant benefits over conventional surgical tools. The configuration of the actuating means described above such as embodiments comprising a bender transducer actuator, cymbal transducer/actuator actuator, Langevin actuator 311 actuator or an APA transducer actuator accommodates the use of piezoelectric actuating members in a surgical instrument by enabling the displacement of the cutting member or blade to such velocities that cause a reduction of force needed for cutting, incising, or penetrating of tissue during surgical procedures. Electrical signal control facilitated by an electrically coupled feedback system could provide the capability of high cut rate actuation, control over cut width, and low traction force for these procedures.

Now that exemplary embodiments of the present invention have been shown and described in detail, various modifications and improvements thereon will become readily apparent to those skilled in the art. While the foregoing embodiments may have dealt with the incision of an eyeball as an exemplary biological tissue, the present invention can undoubtedly ensure similar effects with other tissues commonly incised during surgery. For example there are multiplicities of other applications like restorative or reconstructive microsurgery, cardiology or neurology, to name a few, where embodiments disclosed herein comprising sonically or ultrasonically driven cutting edges may be used to precisely pierce or incise tissues other than that forming an eyeball. Furthermore, while the previous embodiments have relied heavily on examples in which the surgical blades are vibrated sinusoidally in a direction parallel to the surface of the tissue or material being incised, cut, divided or penetrated by the blade, they are not limited to such locomotion in such a relative direction. For example, the motion of the blades of the previously described embodiments may actually be sinusoidal and in a direction that is perpendicular to the surface of the tissue or material being incised, cut, divided or penetrated by the blade. Accordingly, the spirit and scope of the present invention is to be construed broadly and limited only by the appended claims, and not by the foregoing specification.

The invention claimed is:

1. A surgical cutting device comprising:
a body; a piezoelectric actuator received within and secured to the body; a blade associated with and in communication with said actuator, said actuator adapted for vibrating at a frequency to produce an oscillating displacement of the blade.

2. A surgical cutting device of claim 1 wherein said actuator is adapted for vibrating at a frequency to produce a sinusoidal displacement of the blade.

3. The surgical cutting device of claim 1, wherein the piezoelectric actuator comprises a support bar having a proximal end and a distal end, said actuator further comprising a first surface and a second surface; and at least one piezoelectric ceramic plate attached to one of said first surface and said second surface of said support bar; said distal end of said support bar being fixedly attached to an inner wall portion of said body by a motion constraint; and wherein said blade comprises a collar portion attached to the proximal end of said support bar.

4. The surgical cutting device of claim 3, wherein said blade comprises a tip, a first blade ear, a second blade ear, a first cutting edge surface between said first blade ear and said tip; and a second cutting edge surface between said second blade ear and said tip.

5. The surgical cutting device of claim 4 wherein said second blade ear and said tip are formed essentially on the same plane; and wherein said first ear corresponds to a same side of a central portion of the device as said first surface of said actuator; and wherein said second blade ear corresponds with said second surface of said actuator at an opposite side of said central portion of the device.

6. The surgical cutting device of claim 3 wherein the actuator is of a variable thickness.

7. The surgical cutting device of claim 1 wherein the actuator is a cymbal transducer/actuator.

8. The surgical cutting device of claim 1 wherein the actuator is a Langevin actuator 311.

9. The surgical cutting device of claim 1 wherein the actuator is an amplified piezoelectric actuator.

10. The surgical cutting device of claim 1 wherein said actuator is adapted for vibrating said blade at a peak velocity in the range of 0.9-2.5 m/s.

11. The surgical cutting device of claim 1 wherein said actuator is adapted for vibrating said blade at a peak velocity in the range of 1.0-2.25 m/s.

12. The surgical cutting device of claim 1 wherein said actuator is adapted for vibrating said blade at a peak velocity in the range of 1.5-2.0 m/s.

13. A method of operating a surgical device comprising:
electrically driving a piezoelectric actuator disposed within and secured to a device body, said electrically driving of the piezoelectric actuator occurring electrically with an AC signal; and
associating said piezoelectric actuator with a blade and causing said blade to oscillate at an equivalent frequency as said AC signal.

14. The method of claim 13 wherein electrically driving of the piezoelectric actuator occurs electrically with an AC signal at an electric field of between 300-500 V/mm and at a frequency of 450 Hz.

15. The method of claim 14 wherein said displacement is in the range of 250-500 μm.

16. The method of claim 13 wherein said actuator is adapted for vibrating at a frequency to produce a sinusoidal displacement of the blade.

17. The method of claim 16 wherein during the sinusoidal displacement, said blade has a peak velocity in the range of 0.9-2.5 m/s.

18. The method of claim 16 wherein during the sinusoidal displacement, said blade has a peak velocity in the range of 1.0-2.25 m/s.

19. The method of claim 16 wherein during the sinusoidal displacement, said blade has a peak velocity in the range of 1.5-2.0 m/s.

20. A method of operating a surgical device comprising:
providing a surgical cutting device having a body, a piezoelectric actuator received within and secured to the body, and a blade associated with and in communication with said actuator;
electrically driving said piezoelectric actuator, said electrically driving of the piezoelectric actuator occurring electrically with an AC signal and causing said blade to oscillate at an equivalent frequency as said AC signal.

21. The method of claim 20 wherein said actuator is adapted for vibrating at a frequency to produce a sinusoidal displacement of the blade in the range of 250-500 μm.
